(19) United States
(12) Patent Application Publication
(10) Pub. No.: US 2020/0077871 A1
(43) Pub. Date: Mar. 12, 2020

(54) ENDOSCOPIC SYSTEM

(71) Applicant: Olympus Corporation, Tokyo (JP)

(72) Inventors: Tomoharu Ogihara, Tokyo (JP); Keisuke Tsutsui, Kawaguchi-shi (JP)

(73) Assignee: Olympus Corporation, Tokyo (JP)

(21) Appl. No.: 16/682,107

(22) Filed: Nov. 13, 2019

Related U.S. Application Data
(63) Continuation of application No. PCT/JP2018/014620, filed on Apr. 5, 2018.

(30) Foreign Application Priority Data
May 18, 2017 (JP) 2017-99044

(51) Int. Cl. A61B 1/00; A61B 1/045

(52) U.S. Cl. CPC A61B 1/00036; A61B 1/045; A61B 1/00009; A61B 1/00006; A61B 1/00011

(57) ABSTRACT

An endoscopic system comprises an image capturing device. The image capturing device captures an image of an examinee and generates a captured image signal with respect to the examinee. A control device is connected to the endoscope. A first controller is disposed in the endoscope and having a first sender/receiver communicating with the control device during a predetermined period when the endoscope is connected to the control device so as to send a plurality of parameters for driving the image capturing device to the control device. A second controller is disposed in the control device and having a second sender/receiver receiving the parameters sent from the first controller during the predetermined period when the endoscope is connected to the control device. An output value controller is disposed in the control device and setting predetermined output values relative to the control device required to drive the image capturing device.

### CROSS-REFERENCE TO RELATED APPLICATIONS

This application is a continuation application of PCT Application No. PCT/JP2018/014620 filed on Apr. 5, 2018, which in turn claim priority to the Japanese Patent Application No. 2017-99044 filed on May 18, 2017 in Japan which is hereby incorporated by reference in its entirety.

### TECHNICAL FIELD

The disclosed technology relates to an endoscopic system, and more particularly to an endoscopic system including an endoscope that has an image capturing device and an image processing apparatus that includes a power supply unit supplying a power supply voltage to the endoscope.

### DESCRIPTION OF THE RELATED ART

Endoscopic systems have been widely used in the medical field, the industrial field, etc. Each of the endoscopic systems includes an endoscope that has an image capturing device capturing an image of a subject in an examinee and an image processing apparatus, referred to as a video processor, generating an observational image of the subject based on the image captured by the endoscope.

For use as the endoscopes of such endoscopic systems, there have been widely used endoscopes each employing a charge-coupled device (CCD) image sensor, for example, as the image capturing device, that outputs a captured image signal to be transmitted to an image processing apparatus, i.e., a video processor, at a subsequent stage.

For use as the above image processing apparatus, i.e., the video processor, there has been known a video processor including a driver that sends a predetermined control signal, e.g., a CCD drive pulse signal, to an image capturing device of an endoscope connected thereto, and a power supply unit that supplies a predetermined power supply voltage.

Furthermore, with respect to video processors of the type described, there is also known a technology for detecting the kind of an image capturing device of an endoscope connected thereto, and controlling the endoscope so as to optimally drive the image capturing device according to the detected kind.

There is known a technology, with respect to the video processors described hereinbefore that have a function to determine image capturing devices, for determining the type of an endoscope, i.e., an image capturing device, by measuring a resistor disposed in a connector of the connected endoscope, for example.

There is also known a technology in which an identification (ID) memory storing ID information on an endoscope, i.e., an image capturing device, is included in a connector or the like of the endoscope, and a video processor determines the type of the image detecting device based on the information stored in the ID memory at the time the endoscope is connected to the video processor as disclosed in Japanese Patent Application-JP 2010-88656 A.

More specifically, according to the technology disclosed in JP 2010-88656 A, image capturing device information is recorded in the ID memory included in the endoscope in advance, and is sent to the video processor when the endoscope is connected to the video processor. Thereafter, the video processor determines the image capturing device included in the connected endoscope from a look-up table (LUT) based on the image capturing device information, and controls the power supply unit according to a power supply voltage value for driving the image capturing device.

As far as existing endoscopes are concerned, the endoscopic system disclosed in JP 2010-88656 A can determine the type of an image capturing device from the LUT held in the video processor and drive the image capturing device in a manner suitable for the image capturing device.

However, in order for the endoscopic system disclosed in JP 2010-88656 A to be compatible with all endoscopes, i.e., image capturing devices, that can be connected, the endoscopic system has to keep all image capturing device information. It is difficult for the endoscopic system to realize such information storage, i.e., to determine a vast number of types of image capturing devices.

Furthermore, in the first place, the LUT in the video processor lacks information on the image capturing devices of new endoscopes that have not yet been available in the market. Consequently, the endoscopic system disclosed in JP 2010-88656 A finds it difficult to select an optimum driving process for a new endoscope that has not yet been available in the market.

### BRIEF SUMMARY OF EMBODIMENTS

One aspect of the disclosed technology is directed to an endoscopic system comprises an endoscope having an image capturing device. The image capturing device captures an image of an examinee and generates a captured image signal with respect to the examinee. A control device is connected to the endoscope. A first controller is disposed in the endoscope and having a first sender/receiver communicating with the control device during a predetermined period when the endoscope is connected to the control device so as to send a plurality of parameters for driving the image capturing device to the control device. A second controller is disposed in the control device and having a second sender/receiver receiving the parameters sent from the first controller during the predetermined period when the endoscope is connected to the control device. An output value controller is disposed in the control device and setting predetermined output values relative to the control device required to drive the image capturing device. The parameters have a plurality of predetermined communication formats for driving the image capturing device and the output value controller sets respective values of the parameters entered according to the predetermined communication formats as the output values.

The control device includes a power supply unit generating electric power to drive the image capturing device. A plurality of power supply voltage generators generating predetermined power supply voltages that are different from one another, which are required to drive the image capturing device in response to an output voltage from the power supply unit. An overcurrent detector detecting overcurrents relative to output electric currents from the plurality of power supply voltage generators. A clock generator generating a drive clock for driving the image capturing device. The output value controller includes a power supply voltage setting unit setting voltage values for the predetermined power supply voltages to be generated by the plurality of power supply voltage generators. A power supply sequence setting unit setting a sequence timing to turn on and off the power supply voltages for the plurality of power supply voltage generators. An overcurrent detection threshold value setting unit setting an overcurrent detection threshold value for the overcurrent detector to detect overcurrents and a clock frequency setting unit setting a frequency for the drive clock to be generated by the clock generator. The power supply voltage setting unit, the power supply sequence setting unit, the overcurrent detection threshold value setting unit, and the clock frequency setting unit of the output value controller all of which are included in the second controller. The clock frequency setting unit of the output value controller is included in the second controller, and the power supply voltage setting unit, the power supply sequence setting unit, and the overcurrent detection threshold value setting unit of the output value controller are not included in the second controller. The power supply voltage setting unit, the power supply sequence setting unit, and the overcurrent detection threshold value setting unit of the output value controller are included in the second controller, and the clock frequency setting unit of the output value controller is not included in the second controller. The power supply voltage setting unit, the power supply sequence setting unit, the overcurrent detection threshold value setting unit, and the clock frequency setting unit of the output value controller are not included in the second controller. The predetermined period includes a period that the image capturing device operates normally when the endoscope is connected to the control device.

Another aspect of the disclosed technology is directed to an endoscope used in an endoscopic system. The endoscope comprises an image capturing device being disposed in the endoscope and configured to capture an image of an examinee and to generate a captured image signal with respect to the examinee. A first controller is disposed in the endoscope and having a first sender/receiver communicating with a control device during a predetermined period when the endoscope is connected to the control device, to send a plurality of parameters having a plurality of predetermined communication formats for driving the image capturing device to the control device.

A further aspect of the disclosed technology is directed to an endoscope having a control device connected thereto. The control device comprises a second controller being disposed in the control device and having a second sender/receiver receiving a plurality of parameters having a plurality of predetermined communication formats for driving an image capturing device in an endoscope which are sent from the endoscope during a predetermined period when the endoscope is connected to the control device. And an output value controller setting respective values of the received parameters entered according to the predetermined communication formats as output values for driving the image capturing device.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a view illustrating a configuration of an endoscopic system according to a first embodiment of the disclosed technology.

FIG. 2 is a block diagram illustrating an electric arrangement of the endoscopic system according to the first embodiment.

FIG. 3 is a diagram illustrating an example of power supply voltage setting information to be sent from an endoscope to a video processor in the endoscopic system according to the first embodiment.

FIG. 4 is a diagram illustrating an example of drive clock frequency setting information to be sent from the endoscope to the video processor in the endoscopic system according to the first embodiment.

FIG. 5 is a block diagram illustrating an electric arrangement of an endoscopic system including an endoscope according to a second embodiment of the disclosed technology.

FIG. 6 is a block diagram illustrating an electric arrangement of an endoscopic system including an endoscope according to a third embodiment of the disclosed technology.

FIG. 7 is a block diagram illustrating an electric arrangement of an endoscopic system including an endoscope according to a fourth embodiment of the disclosed technology.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

Embodiments of the disclosed technology will hereinafter be described hereinafter with reference to the drawings.

It is an object of the disclosed technology to provide an endoscopic system capable of optimally driving new endoscopes to be introduced into the market in addition to existing endoscopes.

According to an aspect of the disclosed technology, there is provided an endoscopic system having an endoscope including an image capturing device, and a control device connected to the endoscope, the endoscopic system including the image capturing device which captures an image of an examinee and generates a captured image signal with respect to the examinee, a first controller disposed in the endoscope and having a first sender/receiver communicating with the control device during a predetermined period until the image capturing device operates normally when the endoscope is connected to the control device to send parameters having a plurality of predetermined formats for driving the image capturing device to the control device, a second controller disposed in the control device and having a second sender/receiver receiving the parameters sent from the first controller during the predetermined period when the endoscope is connected to the control device, and an output value controller disposed in the control device and setting predetermined output values relative to the control device required to drive the image capturing device using the parameters received by the second sender/receiver.

### First Embodiment

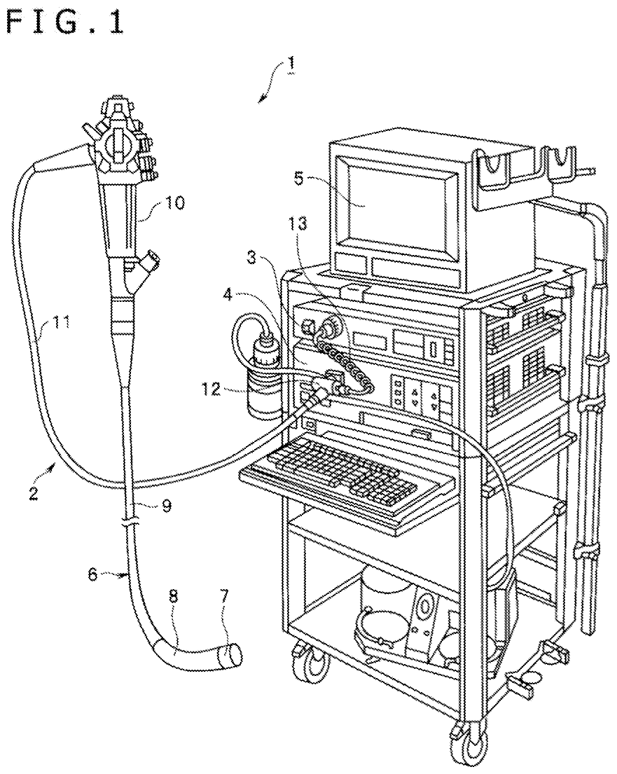

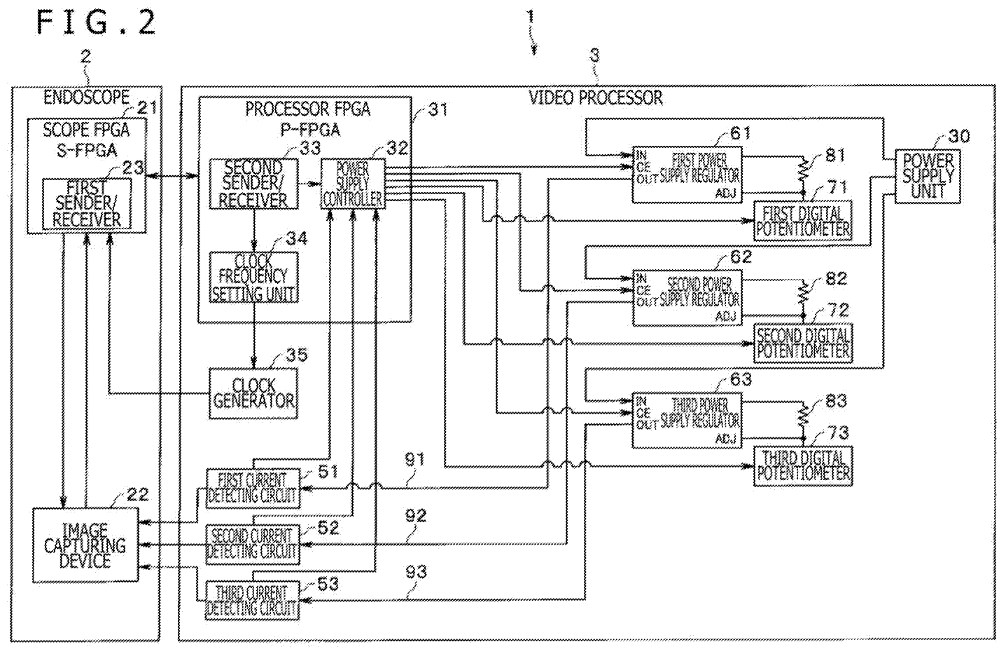

FIG. 1 is a view illustrating a configuration of an endoscopic system according to a first embodiment of the disclosed technology, and FIG. 2 is a block diagram illustrating an electric arrangement of the endoscopic system according to the first embodiment.

As illustrated in FIGS. 1 and 2, an endoscopic system 1 according to the first embodiment has an endoscope 2 observing and capturing images of an examinee, a video processor 3 connected to the endoscope 2 and performing a predetermined image processing process on a captured image signal input from the endoscope 2, a light source device 4 supplying illuminating light to illuminate the examiner, and a monitor device 5 displaying an observational image based on the captured image signal.

Configuration of the Endoscope 2

The endoscope 2 has an insertion portion 6 to be inserted into a body cavity etc. of the examinee, an endoscope operating portion 10 disposed on a proximal-end side of the insertion portion 6 and gripped by a surgeon for operating the endoscope 2, and a universal cord 11 having an end disposed such that the universal cord 11 extends from a side of the endoscope operating portion 10.

The insertion portion 6 has a hard distal-end portion 7 disposed on a distal-end side thereof, a bendable portion 8 that is freely bendable which is disposed on a rear end of the distal-end portion 7, and a flexible tube portion 9 which is elongate and flexible and disposed on a rear end of the bendable portion 8.

A connector 12 is disposed on a proximal-end side of the universal cord 11 and connected to the light source device 4. Specifically, a pipe sleeve, not illustrated, as a joint end of a fluid tube that projects from a distal end of the connector 12 and a light guide pipe sleeve, not illustrated, as a supply end for illumination light are detachably connected to the light source device 4.

A connection cable 13 has an end connected to an electric contact disposed on a side of the connector 12. The connection cable 13 includes a signal line for transmitting image signals from an image capturing device 22, or a CCD image sensor (see FIG. 2), in the endoscope 2, for example. The connection cable 13 has, on another end thereof, a connector connected to the video processor 3.

The connector 12 houses therein an analog front end (AFE), not illustrated, an endoscopic field programmable gate array (FPGA) 21, or a scope FPGA, a storage unit, not illustrated, storing inherent ID information of the endoscope 2, and the like. The scope FPGA 21 will be described in detail hereinafter.

As illustrated in FIG. 2, the endoscope 2 includes an objective optical system, not illustrated, disposed in the distal-end portion 7 of the insertion portion 6 and including a lens for introducing a subject image, and an image capturing device 22, or a CCD image sensor, disposed on a focusing plane in the objective optical system.

According to the present embodiment, the image capturing device 22 is a solid-state image capturing device functioning as a CCD image sensor, as described hereinbefore. The image capturing device 22 generates a captured image signal from a subject image by way of photoelectric conversion and outputs the generated captured image signal to a subsequent stage.

According to the present embodiment, the image capturing device 22 is supplied with a plurality of power supply voltages generated by the video processor 3, e.g., a digital-system power supply voltage of 1 V, an interface-system power supply voltage of 2 V, and an analog-system power supply voltage of 3 V. The image capturing device 22 is driven by a predetermined drive clock pulse signal sent from the video processor 3 as well.

Furthermore, the endoscope 2 includes the scope FPGA 21, hereinafter referred to as “S-FPGA 21,” disposed in the connector 12. The S-FPGA 21 is configured as a so-called FPGA, and functions as a timing pulse adjuster for making various timing adjustments under the control of the video processor 3 and also as a first sender/receiver 23 according to the present embodiment.

The first sender/receiver 23 functions as the first sender/receiver which communicates with a second sender/receiver 33, to be described hereinafter, in the video processor 3 during a predetermined period until the image capturing device 22 operates normally when the endoscope 2 is connected to the video processor 3, and sends a plurality of parameters having predetermined formats for driving the image capturing device 22 to the second sender/receiver 33 in the video processor 3.

The timing pulse adjuster described hereinabove in the S-FPGA 21 receives drive clock pulses generated by a clock generator 35 in the video processor 3 and sends various timing pulse signals relative to the driving of the image capturing device 22 to the image capturing device 22.

The S-FPGA 21 functions as a first controller having the first sender/receiver. Operation and advantages of the S-FPGA 21 and a processor FPGA (P-FPGA) 31 in the video processor 3, to be described hereinafter, will be described in detail hereinafter.

Configuration of the Video Processor 3

Referring back to FIG. 2, the endoscopic system 1 according to the present embodiment includes the video processor 3 connected to the endoscope 2 and performing a predetermined image processing process on a captured image signal input from the endoscope 2.

According to the present embodiment, the video processor 3 is a control device connected to the endoscope, and includes known circuits, not illustrated, such as an image processing unit performing a predetermined image processing process on a captured image signal input from the endoscope 2, a video output unit outputting a captured image signal processed by the image processing unit to the monitor device 5 (see FIG. 1), an operation control unit sending various operation control signals to the endoscope 2, and so on.

Also, according to the present embodiment, as illustrated in FIG. 2, the video processor 3 includes a power supply unit 30 generating power supply voltages to be supplied to the various circuits in the video processor 3 and power supply voltages to be supplied to the various circuits, including the image capturing device 22, etc., in the endoscope 2, a plurality of power supply regulators 61, 62, and 63 generating various power supply voltages to be supplied to the image capturing device 22, a plurality of current detecting circuits 51, 52, and 53 detecting output electric currents output from the power supply regulators, and the clock generator 35 that generates drive clock pulses for driving the image capturing device 22.

Furthermore, the video processor 3 includes the processor FPGA (P-FPGA) 31 that sends signals to and receives signals from the S-FPGA 21 in the endoscope 2 and that controls the circuits in the video processor 3.

The processor FPGA 31, hereinafter referred to as P-FPGA 31, is configured as a so-called FPGA, and functions as a circuit for generating various control signals to be sent to the endoscope 2 and a control circuit for the various circuits in the video processor 3.

Also, according to the first embodiment, the P-FPGA 31 functions as a clock frequency setting unit 34 for controlling the clock generator 35, the second sender/receiver 33 that receives the parameters sent from the S-FPGA 21 in the endoscope 2 during the predetermined period when the endoscope 2 is connected to the video processor 3, and a power supply controller 32 for controlling the power supply regulators 61, 62, and 63, the current detecting circuits 51, 52, and 53, the clock frequency setting unit 34, etc. according to the received parameters.

The P-FPGA 31 also functions as a second controller as the second sender/receiver that receives predetermined parameter information from the first sender/receiver 23 in the S-FPGA 21 in the endoscope 2, and also as an output value controller that sets predetermined output values relative to the control device, i.e., the video processor 3, required to drive the image capturing device 22. Operation and advantages of those functions of the P-FPGA 31 and the S-FPGA 21 in the endoscope 2 will be described in detail hereinafter.

A: Power Supply Voltage Control in the Video Processor 3

As illustrated in FIG. 2, the video processor 3 according to the present embodiment has the first power supply regulator 61, the second power supply regulator 62, and the third power supply regulator 63 that generate and output various power supply voltages V1, V2, and V3 in response to predetermined power supply voltages from the power supply unit 30.

The first power supply regulator 61 generates a predetermined first voltage V1 in response to a power supply voltage from the power supply unit 30 and supplies the generated first voltage V1 as a first regulator output Vol1 through a first power supply line 91 and the first current detecting circuit 51 to the image capturing device 22. According to the present embodiment, the first voltage V1 is assumed to be a voltage of 3 V for an analog power supply (ANA).

Similarly, the second power supply regulator 62 generates a predetermined second voltage V2 in response to a power supply voltage from the power supply unit 30 and supplies the generated second voltage V2 as a second regulator output Vol2 through a second power supply line 92 and the second current detecting circuit 52 to the image capturing device 22. According to the present embodiment, the second voltage V2 is assumed to be a voltage of 2 V for an interface power supply (IF).

Similarly, the third power supply regulator 63 generates a predetermined third voltage V3 in response to a power supply voltage from the power supply unit 30 and supplies the generated third voltage V3 as a third regulator output Vol3 through a third power supply line 93 and the third current detecting circuit 53 to the image capturing device 22. According to the present embodiment, the third voltage V3 is assumed to be a voltage of 1 V for a digital power supply (DIG).

The first power supply regulator 61, the second power supply regulator 62, and the third power supply regulator 63 function as a plurality of power supply voltage generators generating different predetermined power supply voltages required to drive the image capturing device 22 in response to the output voltages from the power supply unit 30.

Meanwhile, the first power supply regulator 61 has an ADJ terminal to which an external fixed resistor 81 and a first digital potentiometer 71 are connected. According to the present embodiment, the first digital potentiometer 71 has its resistance value variable under the control of the power supply controller 32.

The first power supply regulator 61 to which the first digital potentiometer 71 is connected can variably control its first regulator output Vol1 from the first power supply regulator 61 by having the resistance value of the first digital potentiometer 71 appropriately changed under the control of the power supply controller 32.

According to the present embodiment, based on the feature that the first regulator output Vol1 from the first power supply regulator 61 is variably controlled by controlling the resistance value of the first digital potentiometer 71, as described hereinbefore, the power supply controller 32 controls the resistance value of the first digital potentiometer 71 according to the predetermined parameter information sent from the S-FPGA 21 in the endoscope 2, thereby controlling the output value of the first power supply regulator 61. This operation will be described in detail hereinafter.

Meanwhile, the second power supply regulator 62 has an ADJ terminal to which an external fixed resistance 82 and a second digital potentiometer 72 are connected, as with the case described hereinabove. Furthermore, the third power supply regulator 63 has an ADJ terminal to which an external fixed resistor 83 and a third digital potentiometer 73 are connected, as with the case described herein above.

Each of the second digital potentiometer 72 and the third digital potentiometer 73 has its resistance value variable under the control of the power supply controller 32, as is the case with the first digital potentiometer 71.

The second power supply regulator 62 to which the second digital potentiometer 72 is connected and the third power supply regulator 63 to which the third digital potentiometer 73 is connected can variably control the second regulator output Vol2 from the second power supply regulator 62 and the third regulator output Vol3 from the third power supply regulator 63, respectively, by having the resistance values of the second digital potentiometer 72 and the third digital potentiometer 73 appropriately changed under the control of the power supply controller 32.

In addition, as with the case described hereinabove, the power supply controller 32 controls the resistance values of the second digital potentiometer 72 and the third digital potentiometer 73 according to the predetermined parameter information sent from the S-FPGA 21 in the endoscope 2, thereby controlling the output values of the second power supply regulator 62 and the third power supply regulator 63. This operation will also be described in detail hereinafter.

B: Power Supply Sequence Control in the Video Processor 3

Next, power supply sequence control in the video processor 3 will be described hereinafter.

As described hereinbefore, the image capturing device 22 according to the present embodiment is supplied with the plurality of power supply voltages from the plurality of power supply voltage generators, i.e., the first power supply regulator 61, the second power supply regulator 62, and the third power supply regulator 63.

Also, in these types of a plurality of power supply regulators, since the image capturing device 22 etc. may possibly suffer a failure unless these power supply regulators strictly follow a proper power supply sequence, the power supply sequence is generally strictly controlled.

According to the present embodiment, each of the first power supply regulator 61, the second power supply regulator 62, and the third power supply regulator 63 has a chip enable function, and is enabled and disabled under control of the power supply controller 32.

Specifically, the first power supply regulator 61, the second power supply regulator 62, and the third power supply regulator 63 have respective CE terminals supplied with CE control signals from the power supply controller 32, and are enable and disabled under the control of the power supply controller 32.

C: Overcurrent Detection in the Video Processor 3

Next, overcurrent detection control in the video processor 3 will be described hereinafter.

As described hereinbefore, the video processor 3 according to the present embodiment has the first current detecting circuit 51 that detects a first electric current I1 on the first power supply line 91 connected to the first power supply regulator 61, the second current detecting circuit 52 that detects a second electric current I2 on the second power supply line 92 connected to the second power supply regulator 62, and the third current detecting circuit 53 that detects a third electric current I3 on the third power supply line 93 connected to the third power supply regulator 63.

The first current detecting circuit 51, the second current detecting circuit 52, and the third current detecting circuit 53 have respective output terminals connected to the image capturing device 22 in the endoscope 2, so that the first regulator output Vol1 form the first power supply regulator 61, the second regulator output Vol2 from the second power supply regulator 62, and the third regulator output Vol3 from the third power supply regulator 63 are supplied to the image capturing device 22.

Moreover, the first current detecting circuit 51, the second current detecting circuit 52, and the third current detecting circuit 53 detect electric current values respectively on the first power supply line 91, the second power supply line 92, and the third power supply line 93 and send the detected results to the power supply controller 32 under the control of the power supply controller 32.

Furthermore, according to the present embodiment, the power supply controller 32 in the P-FPGA 31 has input terminals to which the detected results, i.e., the detected electric current values including a first electric current, a second electric current, and a third electric current, from the first current detecting circuit 51, the second current detecting circuit 52, and the third current detecting circuit 53 are input. The input terminals are combined with respective AD converters for converting the input electric current values into digital electric current values.

The detected electric current values that have been converted into the digital electric current values by the AD converters in the power supply controller 32 are compared with a predetermined overcurrent detection threshold value by a comparator in the power supply controller 32, so that overcurrents with respect to the detected electric current values including the first electric current, the second electric current, and the third electric current can be detected.

D: Frequency Setting for Drive Clock Pulses in the Video Processor 3

Next, frequency setting for drive clock pulses in the video processor 3 will be described hereinafter.

As described hereinbefore, the video processor 3 includes the clock generator 35 that generates a drive clock for driving the image capturing device 22. According to the first embodiment, the P-FPGA 31 includes the clock frequency setting unit 34 for controlling the clock generator 35, and the clock frequency setting unit 34 sets a frequency for the drive clock pulses.

Operation and Advantages of the S-FPGA 21 and the P-FPGA 31

Next, operation and advantages of the scope FPGA 21, or S-FPGA, disposed in the endoscope 2 and the processor FPGA 31, or P-FPGA, disposed in the video processor 3 according to the present embodiment will be described in detail hereinafter.

As described hereinbefore, each of the S-FPGA 21 and the P-FPGA 31 is configured as an FPGA, with the S-FPGA 21 including the first sender/receiver 23 and the P-FPGA 31 including the second sender/receiver 33.

The first sender/receiver 23 in the S-FPGA 21 and the second sender/receiver 33 in the P-FPGA 31 communicate with each other during a predetermined period until the image capturing device 22 operates normally when the endoscope 2 is connected to the video processor 3. During this period, the first sender/receiver 23 sends a plurality of parameters having predetermined formats for driving the image capturing device 22 to the second sender/receiver 33.

The second sender/receiver 33 that has received the “parameters having predetermined formats” sends their information to the power supply controller 32 or the clock frequency setting unit 34, which sets predetermined output values regarding the video processor 3, or the control device, required for driving the image capturing device 22, using the parameters according to the type of the parameters received by the second sender/receiver 33.

The “parameters having predetermined formats for driving the image capturing device 22” described hereinbefore, and the “setting of the predetermined output values” by the power supply controller 32 or the clock frequency setting unit 34 according to the present first embodiment will be described by way of example hereinafter.

First Parameter: Resistance Values for Setting Power Supply Voltages

According to the first embodiment, as described hereinbefore, the first power supply regulator 61, the second power supply regulator 62, and the third power supply regulator 63 that generate the three power supply voltages V1, V2, and V3 to be supplied to the image capturing device 22 in the endoscope 2 are included in the video processor 3.

As described hereinbefore, according to the present embodiment, these three power supply voltages are assumed to be a voltage of 3 V for an analog power supply (ANA), a voltage of 2 V for an interface power supply (IF), and a voltage of 1 V for a digital power supply (DIG).

Furthermore, as described hereinbefore, the first power supply regulator 61, the second power supply regulator 62, and the third power supply regulator 63 are combined with the first digital potentiometer 71, the second digital potentiometer 72, and the third digital potentiometer 73, respectively. As described hereinbefore, it is possible to control the output values, i.e., the power supply voltage values, from the power supply regulators by controlling the “resistance values” regarding the potentiometers.

In the endoscopic system 1 according to the first embodiment, information on the “resistance values” for setting the power supply voltages is set as the first parameter of the “parameters having predetermined formats for driving the image capturing device 22.”

In the endoscopic system 1 according to the first embodiment, specifically, the “resistance value” information regarding the digital potentiometers is sent as first parameter information from the first sender/receiver 23 in the endoscope 2 to the second sender/receiver 33 in the video processor 3 by the communication in the predetermined period between the S-FPGA 21 in the endoscope 2 and the P-FPGA 31 in the video processor 3.

The second sender/receiver 33 in the P-FPGA 31 that has received the “resistance value” information as the first parameter sends the “resistance value” information to the power supply controller 32 in the same P-FPGA 31.

Based on the received “resistance value” information, the power supply controller 32 controls the “resistance value” regarding the digital potentiometer connected to the power supply regulator corresponding to the information, i.e., either one of the power supply regulators including the first power supply regulator 61, the second power supply regulator 62, or the third power supply regulator 63, or in other words, either one of the digital potentiometers including the first digital potentiometer 71, the second digital potentiometer 72, or the third digital potentiometer 73.

In this manner, the output value, or the power supply voltage value, of the power supply regulator corresponding to the “resistance value” information is controlled so as to be changed.

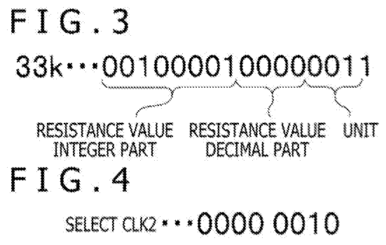

FIG. 3 is a diagram illustrating an example of power supply voltage setting information to be sent from the endoscope to the video processor in the endoscopic system according to the first embodiment.

More specifically, in a case where the “resistance value” regarding the digital potentiometer is 33 kΩ, for example, information of a resistance value integer part, a resistance value decimal part, and a unit, as illustrated in FIG. 3 is sent as serial data from the first sender/receiver 23 in the S-FPGA 21 to the second sender/receiver 33 in the P-FPGA 33.

Second Parameter: A Setting Value for the Power Supply Sequence

According to the first embodiment, the first power supply regulator 61, the second power supply regulator 62, and the third power supply regulator 63 are included in the video processor 3, and since the image capturing device 22 etc. may possibly suffer a failure unless these power supply regulators strictly follow a proper power supply sequence, the power supply sequence is strictly controlled, as described hereinbefore.

Specifically, according to the first embodiment, the first power supply regulator 61, the second power supply regulator 62, and the third power supply regulator 63 are supplied with respective CE control signals from the power supply controller 32, and are enabled and disabled by under the control of the power supply controller 32, i.e., a power supply sequence is controlled by the power supply controller 32.

In the endoscopic system according to the first embodiment, information of a “sequence setting value” of the power supply sequence is set as a second parameter of the “parameters having predetermined formats for driving the image capturing device 22.”

In the endoscopic system 1 according to the first embodiment, specifically, the “sequence setting value” information regarding the power supply sequence is sent as second parameter information from the first sender/receiver 23 in the endoscope 2 to the second sender/receiver 33 in the video processor 3 by the communication in the predetermined period between the S-FPGA 21 in the endoscope 2 and the P-FPGA 31 in the video processor 3.

The second sender/receiver 33 in the P-FPGA 31 that has received the “sequence setting value” information as the second parameter sends the “sequence setting value” information to the power supply controller 32 in the same P-FPGA 31.

Based on the received “sequence setting value” information, the power supply controller 32 sends predetermined CE control signals to the power supply regulators to enable or disable the power supply regulators, i.e., the first power supply regulator 61, the second power supply regulator 62, and the third power supply regulator 63, in a sequence corresponding to the information.

The power supply sequence of the first power supply regulator 61, the second power supply regulator 62, and the third power supply regulator 63 is thus appropriately controlled by the CE control signals from the power supply controller 32.

Third Parameter: Overcurrent Detection Threshold Value

According to the first embodiment, as described hereinbefore, the first current detecting circuit 51, the second current detecting circuit 52, and the third current detecting circuit 53 are included to detect the first electric current on the first power supply line 91 connected to the first power supply regulator 61, the second electric current on the second power supply line 92 connected to the second power supply regulator 62, and the third electric current on the third power supply line 93 connected to the third power supply regulator 63.

The detected values, i.e., the detected electric current values including the first electric current, the second electric current, and the third electric current, from the first current detecting circuit 51, the second current detecting circuit 52, and the third current detecting circuit 53 are input to the power supply controller 32, which compares them with a predetermined overcurrent detection threshold value to detect overcurrents, as described hereinbefore.

In the endoscopic system 1 according to the first embodiment, information of the “overcurrent detection threshold value” for detecting overcurrents is set as a third parameter of the “parameters having predetermined formats for driving the image capturing device 22.”

In the endoscopic system 1 according to the first embodiment, specifically, the information of the “overcurrent detection threshold value” for detecting overcurrents is sent as third parameter information from the first sender/receiver 23 in the endoscope 2 to the second sender/receiver 33 in the video processor 3 by the communication in the predetermined period between the S-FPGA 21 in the endoscope 2 and the P-FPGA 31 in the video processor 3.

The second sender/receiver 33 in the P-FPGA 31 that has received the “overcurrent detection threshold value” information sends the “overcurrent detection threshold value” information to the power supply controller 32 in the same P-FPGA 31.

Based on the received “overcurrent detection threshold value” information, the power supply controller 32 sets a predetermined overcurrent detection threshold value with respect to the electric current values detected by the first current detecting circuit 51, the second current detecting circuit 52, and the third current detecting circuit 53.

The power supply controller 32 monitors the electric current values detected by the first current detecting circuit 51, the second current detecting circuit 52, and the third current detecting circuit 53 with respect to the set overcurrent detection threshold value. If the power supply controller 32 detects a overcurrent on either one of the power supply lines including the first power supply line 91, the second power supply line 92, and the third power supply line 93 corresponding to the current detecting circuits, then the power supply controller 32 controls the output of a corresponding one of the first power supply regulator 61, the second power supply regulator 62, and the third power supply regulator 63.

Fourth Parameter: Frequency Setting Value for Drive Clock Pulses

According to the first embodiment, as described hereinbefore, the clock generator 35 generates predetermined drive clock pulses under the control of the clock frequency setting unit 34 in the video processor 3, and supplies the drive clock pulses to the image capturing device 22 in the endoscope 2, i.e., to the S-FPGA 21 in the present embodiment.

In the endoscopic system 1 according to the first embodiment, information of a “frequency setting value” for the drive clock pulses is set as a fourth parameter of the “parameters having predetermined formats for driving the image capturing device 22.”

In the endoscopic system 1 according to the first embodiment, specifically, the “frequency setting value” information regarding the drive clock pulses to be generated by the clock generator 35 is sent as fourth parameter information from the first sender/receiver 23 in the endoscope 2 to the second sender/receiver 33 in the video processor 3 by the communication in the predetermined period between the S-FPGA 21 in the endoscope 2 and the P-FPGA 31 in the video processor 3.

The second sender/receiver 33 in the P-FPGA 31 that has received the “frequency setting value” information as the fourth parameter sends the “frequency setting value” information to the clock frequency setting unit 34 in the same P-FPGA 31.

Based on the received “frequency setting value” information, the clock frequency setting unit 34 controls the clock generator 35 to generate drive clock pulses having a predetermined frequency.

According to the present embodiment, it is assumed that 68 MHz, 74 MHz, and 54 MHz can be set as the frequency of drive clock pulses to be generated by the clock generator 35. These frequencies are assigned to respective given “numeral references,” e.g.,

“CLK1”=68 MHz,

“CLK2”=74 MHz,

“CLK3”=54 MHz.

When the frequency of drive clock pulses desired by the image capturing device 22 in the endoscope 2 connected to the video processor 3 is “74 MHz,” since the corresponding “numeral reference” is “CLK2,” serial data corresponding to “CLK2” are sent as the “frequency setting value” information that is the fourth parameter from the S-FPGA 21 to the P-FPGA 31 according to the present embodiment.

FIG. 4 is a diagram illustrating an example of drive clock frequency setting information to be sent from the endoscope to the video processor in the endoscopic system according to the first embodiment.

As described hereinbefore, since the “numeral reference” corresponding to the frequency “74 MHz” is “CLK2,” the value of “2” that is the integer part of “CLK2” is sent as serial data of the fourth parameter from the S-FPGA 21 to the P-FPGA 31 according to the present embodiment, as illustrated in FIG. 4.

With the endoscopic system 1 according to the first embodiment, as described hereinbefore, when the endoscope 2 and the video processor 3 are connected to each other, the first sender/receiver 23 in the S-FPGA 21 in the endoscope 2 and the second sender/receiver 33 in the P-FPGA 31 in the video processor 3 communicate with each other during a predetermined period until the image capturing device 22 operates normally, i.e., an initial period of the connection. During the predetermined period, a plurality of parameters having predetermined formats for driving the image capturing device 22, i.e., the first through fourth parameters described hereinbefore, are sent from the endoscope 2 to the video processor 3. Therefore, various output values relative to these parameters can properly be set without holding tables of scope information related to the parameters in the video processor 3.

In other words, the endoscopic system 1 according to the first embodiment is capable of optimally driving new endoscopes to be introduced into the market in addition to existing endoscopes.

In the present embodiment, the communication with respect to the parameters described hereinbefore takes place during the predetermined period until the image capturing device 22 operates normally. However, the timing to send the parameters is not limited to the above period, but may be any timing insofar as it is effective to send the parameters.

In the present embodiment, furthermore, the detection of overcurrents on the respective power supply lines is carried out in the power supply controller 32 that receives the detected electric current values sent from the current detecting circuits. The disclosed technology not limited to such details. The first current detecting circuit 51, the second current detecting circuit 52, and the third current detecting circuit 53 described hereinbefore may detect overcurrents themselves, and sent the results of the overcurrent detection to the power supply controller 32. Then, based on overcurrent detection threshold parameter information sent from the endoscope 2, the power supply controller 32 may set a threshold value in the first current detecting circuit 51, the second current detecting circuit 52, and the third current detecting circuit 53.

In the present embodiment, moreover, the parameters sent from the endoscope 2 to the video processor 3 are the first through fourth parameters described hereinbefore. However, the types of parameters are not limited to those parameters, but may be parameters relative to other factors involved in driving the image capturing device 22.

### Second Embodiment

Next, a second embodiment of the disclosed technology will be described hereinafter.

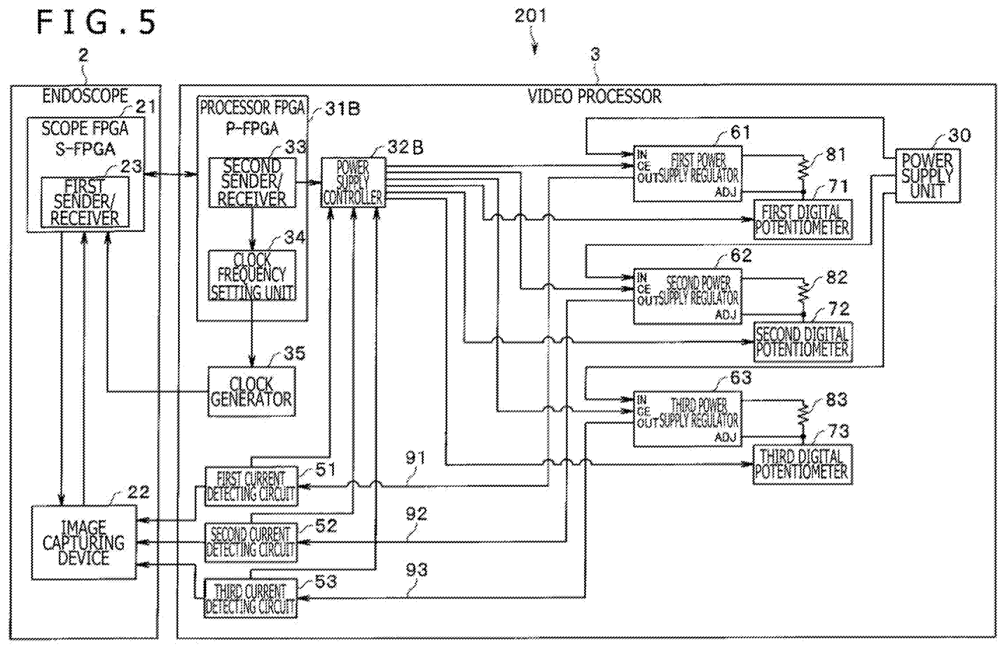

FIG. 5 is a block diagram illustrating an electric arrangement of an endoscopic system including an endoscope according to a second embodiment of the disclosed technology.

The endoscopic system 201 according to the present second embodiment has a basic arrangement that is the same as with the first embodiment. In the endoscopic system 1 according to the first embodiment, both the power supply controller 32 and the clock frequency setting unit 34 are included in the P-FPGA 31. However, the second embodiment has such a feature that that the clock frequency setting unit 34 is included in a P-FPGA 31B, whereas a power supply controller 32B is disposed outside the P-FPGA 31B.

Only the difference with the first embodiment will be described hereinafter, and the common components will not be described hereinafter.

According to the second embodiment, as described hereinbefore, the power supply controller 32B is not included in, but disposed outside, the P-FPGA 31B. However, operation and advantages of the power supply controller 32B are the same as with the first embodiment. Therefore, the endoscopic system 201 according to the second embodiment is also capable of optimally driving new endoscopes to be introduced into the market in addition to existing endoscopes.

### Third Embodiment

Next, a third embodiment of the disclosed technology will be described hereinafter.

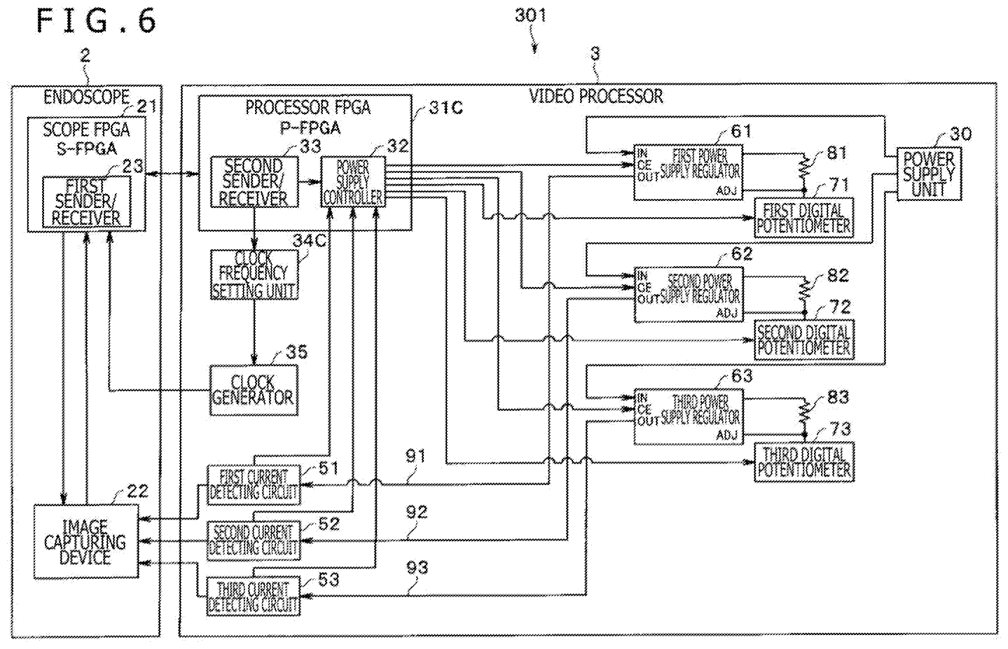

FIG. 6 is a block diagram illustrating an electric arrangement of an endoscopic system including an endoscope according to a third embodiment of the disclosed technology.

The endoscopic system 301 according to the present third embodiment has a basic arrangement that is the same as with the first embodiment. In the endoscopic system 1 according to the first embodiment, both the power supply controller 32 and the clock frequency setting unit 34 are included in the P-FPGA 31. However, the third embodiment has such a feature that the power supply controller 32 is included in a P-FPGA 31C, whereas a clock frequency setting unit 34C is disposed outside the P-FPGA 31C.

Only the difference with the first embodiment will be described hereinafter, and the common components will not be described hereinafter.

According to the third embodiment, as described hereinbefore, the clock frequency setting unit 34C is disposed outside the P-FPGA 31C. However, operation and advantages of the clock frequency setting unit 34C are the same as with the first embodiment. Therefore, the endoscopic system 301 according to the third embodiment is also capable of optimally driving new endoscopes to be introduced into the market in addition to existing endoscopes.

### Fourth Embodiment

Next, a fourth embodiment of the disclosed technology will be described hereinafter.

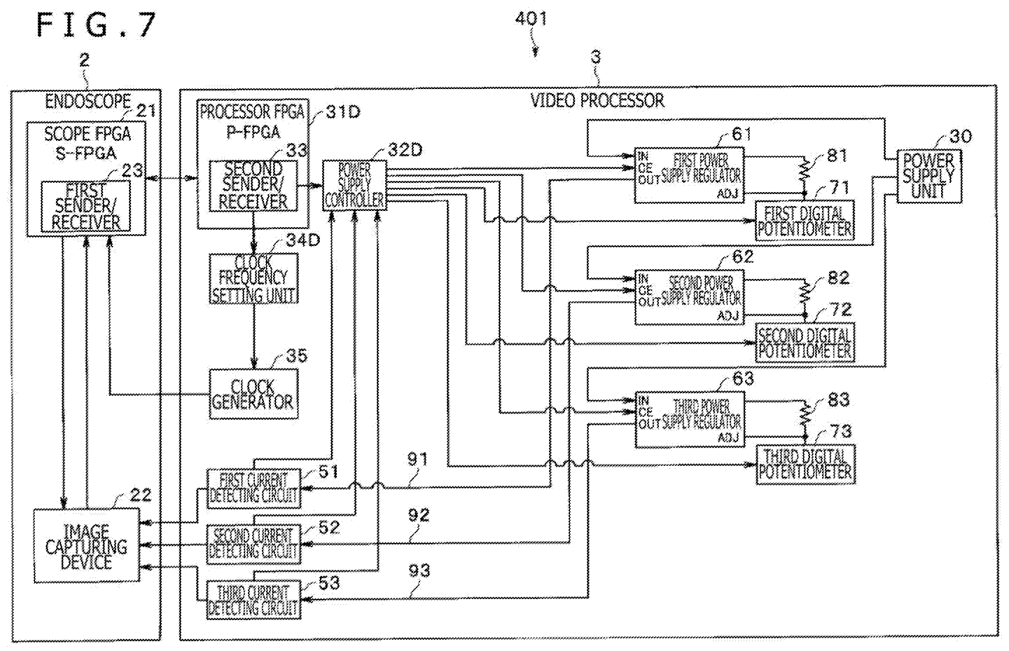

FIG. 7 is a block diagram illustrating an electric arrangement of an endoscopic system including an endoscope according to a fourth embodiment of the disclosed technology.

The endoscopic system 401 according to the present fourth embodiment has a basic arrangement that is the same as with the first embodiment. In the endoscopic system 1 according to the first embodiment, both the power supply controller 32 and the clock frequency setting unit 34 are included in the P-FPGA 31. However, the fourth embodiment has such a feature that both a power supply controller 32D and a clock frequency setting unit 34D are disposed outside a P-FPGA 31D.

Only the difference with the first embodiment will be described hereinafter, and the common components will not be described hereinafter.

According to the fourth embodiment, as described hereinbefore, both the power supply controller 32D and the clock frequency setting unit 34D are disposed outside the P-FPGA 31D. However, operation and advantages of the power supply controller 32D and the clock frequency setting unit 34D are the same as with the first embodiment. Therefore, the endoscopic system 401 according to the fourth embodiment is also capable of optimally driving new endoscopes to be introduced into the market in addition to existing endoscopes.

The disclosed technology is not limited to the embodiments described hereinbefore, but various changes and modifications may be made without departing from the scope of the invention.

In sum, one aspect of the disclosed technology is directed to an endoscopic system comprises an endoscope having an image capturing device. The image capturing device captures an image of an examinee and generates a captured image signal with respect to the examinee. A control device is connected to the endoscope. A first controller is disposed in the endoscope and having a first sender/receiver communicating with the control device during a predetermined period when the endoscope is connected to the control device so as to send a plurality of parameters for driving the image capturing device to the control device. A second controller is disposed in the control device and having a second sender/receiver receiving the parameters sent from the first controller during the predetermined period when the endoscope is connected to the control device. An output value controller is disposed in the control device and setting predetermined output values relative to the control device required to drive the image capturing device. The parameters have a plurality of predetermined communication formats for driving the image capturing device and the output value controller sets respective values of the parameters entered according to the predetermined communication formats as the output values.

The control device includes a power supply unit generating electric power to drive the image capturing device. A plurality of power supply voltage generators generating predetermined power supply voltages that are different from one another, which are required to drive the image capturing device in response to an output voltage from the power supply unit. An overcurrent detector detecting overcurrents relative to output electric currents from the plurality of power supply voltage generators. A clock generator generating a drive clock for driving the image capturing device. The output value controller includes a power supply voltage setting unit setting voltage values for the predetermined power supply voltages to be generated by the plurality of power supply voltage generators. A power supply sequence setting unit setting a sequence timing to turn on and off the power supply voltages for the plurality of power supply voltage generators. An overcurrent detection threshold value setting unit setting an overcurrent detection threshold value for the overcurrent detector to detect overcurrents and a clock frequency setting unit setting a frequency for the drive clock to be generated by the clock generator. The power supply voltage setting unit, the power supply sequence setting unit, the overcurrent detection threshold value setting unit, and the clock frequency setting unit of the output value controller all of which are included in the second controller. The clock frequency setting unit of the output value controller is included in the second controller, and the power supply voltage setting unit, the power supply sequence setting unit, and the overcurrent detection threshold value setting unit of the output value controller are not included in the second controller. The power supply voltage setting unit, the power supply sequence setting unit, and the overcurrent detection threshold value setting unit of the output value controller are included in the second controller, and the clock frequency setting unit of the output value controller is not included in the second controller. The power supply voltage setting unit, the power supply sequence setting unit, the overcurrent detection threshold value setting unit, and the clock frequency setting unit of the output value controller are not included in the second controller. The predetermined period includes a period that the image capturing device operates normally when the endoscope is connected to the control device.

Another aspect of the disclosed technology is directed to an endoscope used in an endoscopic system. The endoscope comprises an image capturing device being disposed in the endoscope and configured to capture an image of an examinee and to generate a captured image signal with respect to the examinee. A first controller is disposed in the endoscope and having a first sender/receiver communicating with a control device during a predetermined period when the endoscope is connected to the control device, to send a plurality of parameters having a plurality of predetermined communication formats for driving the image capturing device to the control device.

A further aspect of the disclosed technology is directed to an endoscope having a control device connected thereto. The control device comprises a second controller being disposed in the control device and having a second sender/receiver receiving a plurality of parameters having a plurality of predetermined communication formats for driving an image capturing device in an endoscope which are sent from the endoscope during a predetermined period when the endoscope is connected to the control device. And an output value controller setting respective values of the received parameters entered according to the predetermined communication formats as output values for driving the image capturing device.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An endoscopic system comprising:
an endoscope having an image capturing device, the image capturing device captures an image of an examinee and generates a captured image signal with respect to the examinee;
a control device being connected to the endoscope;
a first controller being disposed in the endoscope and having a first sender/receiver communicating with the control device during a predetermined period when the endoscope is connected to the control device so as to send a plurality of parameters for driving the image capturing device to the control device;
a second controller being disposed in the control device and having a second sender/receiver receiving the parameters sent from the first controller during the predetermined period when the endoscope is connected to the control device; and
an output value controller disposed in the control device and setting predetermined output values relative to the control device required to drive the image capturing device,
wherein the parameters have a plurality of predetermined communication formats for driving the image capturing device, and
the output value controller sets respective values of the parameters entered according to the predetermined communication formats as the output values.

2. The endoscopic system of claim 1,
wherein the control device includes:
a power supply unit generating electric power to drive the image capturing device;
a plurality of power supply voltage generators generating predetermined power supply voltages that are different from one another, which are required to drive the image capturing device in response to an output voltage from the power supply unit;
an overcurrent detector detecting overcurrents relative to output electric currents from the plurality of power supply voltage generators; and
a clock generator generating a drive clock for driving the image capturing device, and
wherein the output value controller includes:
a power supply voltage setting unit setting voltage values for the predetermined power supply voltages to be generated by the plurality of power supply voltage generators;
a power supply sequence setting unit setting a sequence timing to turn on and off the power supply voltages for the plurality of power supply voltage generators,
an overcurrent detection threshold value setting unit setting an overcurrent detection threshold value for the overcurrent detector to detect overcurrents, and
a clock frequency setting unit setting a frequency for the drive clock to be generated by the clock generator.

3. The endoscopic system of claim 2,
wherein the power supply voltage setting unit, the power supply sequence setting unit, the overcurrent detection threshold value setting unit, and the clock frequency setting unit of the output value controller all of which are included in the second controller.

4. The endoscopic system of claim 2,
wherein the clock frequency setting unit of the output value controller is included in the second controller, and the power supply voltage setting unit, the power supply sequence setting unit, and the overcurrent detection threshold value setting unit of the output value controller are not included in the second controller.

5. The endoscopic system of claim 2,
wherein the power supply voltage setting unit, the power supply sequence setting unit, and the overcurrent detection threshold value setting unit of the output value controller are included in the second controller, and the clock frequency setting unit of the output value controller is not included in the second controller.

6. The endoscopic system of claim 2,
wherein the power supply voltage setting unit, the power supply sequence setting unit, the overcurrent detection threshold value setting unit, and the clock frequency setting unit of the output value controller are not included in the second controller.

7. The endoscopic system of claim 1,
wherein the predetermined period includes a period that the image capturing device operates normally when the endoscope is connected to the control device.

8. An endoscope used in an endoscopic system, the endoscope comprising:
an image capturing device being disposed in the endoscope and configured to capture an image of an examinee and to generate a captured image signal with respect to the examinee; and
a first controller being disposed in the endoscope and having a first sender/receiver communicating with a control device during a predetermined period when the endoscope is connected to the control device, to send a plurality of parameters having a plurality of predetermined communication formats for driving the image capturing device to the control device.

9. An endoscope having a control device connected thereto, the control device comprising:
a second controller being disposed in the control device and having a second sender/receiver receiving a plurality of parameters having a plurality of predetermined communication formats for driving an image capturing device in an endoscope which are sent from the endoscope during a predetermined period when the endoscope is connected to the control device; and
an output value controller setting respective values of the received parameters entered according to the predetermined communication formats as output values for driving the image capturing device.
